(19) United States
(12) Patent Application Publication
(10) Pub. No.: US 2004/0077849 A1
(43) Pub. Date: Apr. 22, 2004

(54) Process for the preparation of cefadroxil

(75) Inventors: Pramod Narayan Deshpande, Chennai (IN); Gautam Kumar Das, Chennai (IN); Rajendra Janardan Sarangdhar, Chennai (IN); Peter Xavier Tharial, Tamilnadu (IN)

Correspondence Address: OLIFF & BERRIDGE, PLC, P.O. BOX 19928, ALEXANDRIA, VA, 22320, US

(73) Assignee: Orchid Chemicals & Pharmaceuticals Limited, Chennai (IN)

(21) Appl. No.: 10/309,692

(22) Filed: Dec. 5, 2002

(30) Foreign Application Priority Data
Oct. 16, 2002 (IN) 760/MAS/2002

(51) Int. Cl. C07D 501/14

(52) U.S. Cl. 540/228

(57) ABSTRACT

The present invention relates to an improved process for the preparation of cefadroxil of the formula (I), more particularly, the present invention relates to an improved process for the preparation of cefadroxil having water content in the range of 4-5%.

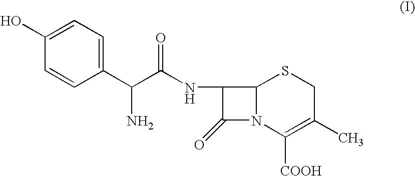

### FIELD OF THE INVENTION

The present invention relates to an improved process for the preparation of cefadroxil of the formula (I). More, particularly, the present invention relates to an improved process for the preparation of cefadroxil having water content in the range of 4-5%.

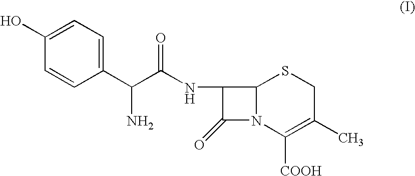

### BACKGROUND OF THE INVENTION

Cefadroxil is chemically known as 7-[D-α-amino-α-(p-hydroxyphenyl)acetamido]-3-methyl-3-cephem-4-carboxylic acid. It is a well-known antibiotic substance having antibacterial activity and is disclosed in U.S. Pat. No. 3,489,752. U.S. Pat. No. 3,985,741 discloses a process for the preparation of cefadroxil by acylation of 7-aminodesacetoxycephalosporanic acid (7-ADCA) with the mixed anhydride of D-(−)-α-(p-hydroxyphenyl)glycine.

U.S. Pat. Nos. 4,160,863 and 4,504,657 discloses a process for the preparation of crystalline cefadroxil monohydrate having a well-defined X-ray diffraction pattern. This crystalline cefadroxil monohydrate is obtained by acylation of silylated 7-ADCA acid with D(−)α-amino-α-(p-hydroxyphenyl)acetyl chloride hydrochloride, cleaving the silyl groups of the acylated product by hydrolysis or alcoholysis, adjusting the pH of the solution with excess DMF to form DMF solvate, dissolving the DMF solvate in acidified water or mixture of acidified water and acetonitrile and precipitating the cefadroxil monohydrate.

U.S. Pat. No. 4,898,938 discloses a method of preparing cefadroxil monohydrate which comprises, slurring cefadroxil solvate with isopropyl alcohol containing from about 6% to 18% of water and isolating the crystalline monohydrate by filtration.

U.S. Pat. No. 4,962,195 discloses yet another novel crystalline cefadroxil having water content of about 3% and characterized by distinct X-ray diffraction properties. This novel cefadroxil is called as “cefadroxil hemihydrate” and is shown to be more stable than crystalline cefadroxil monohydrate.

U.S. Pat. Nos. 4,962,195 and 5,023,331 discloses a method of producing cefadroxil hemihydrate having water content in the range of 2.0 to 3.5% determined by K.F., prepared from dimethylacetamide, monomethylformamide or N-methyl-2-pyrrolidone solvate of cefadroxil, slurring said solvates with a mixture of methanol-isopropyl alcohol 30:70 to 50:50 by volume at a temperature in the range of 45° C. to 55° C., to give crystalline cefadroxil hemihydrate which is isolated by filtration.

Both these patents report that the use of the cefadroxil solvates of dimethylacetamide, N-methyl-2-pyrrolidone and monomethylformamide is critical for the preparation of crystalline cefadroxil hemihydrate.

U.S. Pat. No. 4,358,588 discloses a process for the preparation of cefadroxil comprising silylating 7-ADCA with silylating agent selected from trimethylchlorosilane and treating the resulting silylated ADCA with an equimolar amount of mixed anhydride in the presence of inert anhydrous, organic solvent. This patent discloses the preparation of mixed anhydride of Dane salt.

U.S. Pat. No. 5,998,610 discloses a process for the silylation of 7-ADCA by silylation in certain carboxylic acid esters and its use in the production of 6-alpha-aminoacyl-penicillins and 7-alpha-aminoacyl-desacetoxy-cephalosporins.

U.S. Pat. No. 6,337,396 discloses a method of producing crystalline cefadroxil hemihydrate, from cefadroxil dimethyl formamide solvate which comprises slurrying cefadroxil dimethyl formamide solvate having water content less than 1.8% with a mixture of a lower alkanol and water, at a temperature in the range of about 40° C. to 50° C. and isolating the crystalline cefadroxil hemihydrate from the reaction mixture.

U.S. Pat. No. 5,329,001 disclose a method of producing crystalline cefadroxil having a water content from about 0.8% to about 3.9%.

The above prior art references disclose the preparation of cefadroxil monohydrate or hemihydrate by reacting the silylated 7-ADCA with D(−)α-amino-α-(p-hydroxyphenyl)acetyl chloride hydrochloride, or with mixed anhydride of Dane salt and forming the cefadroxil DMF solvate and desolvating the said DMF solvate using aqueous alcohol. In these processes some amount of alcohol is retained as residual solvent in the final product, which cannot be removed by further purification.

We therefore, focussed our research to have a better process for the preparation of cefadroxil with very specific water content and which gives very good yields. We achieved this by the process of the present invention by using only water for desolvation.

### OBJECTIVE OF THE INVENTION

The primary objective of the present invention is to provide a new method for the preparation of cefadroxil of the formula (I), having water content in the range of 4-5%.

The primary objective of the present invention is to provide a new method for the preparation of cefadroxil of the formula (I), having water content in the range of 4-5%, which is simple and cost effective.

### SUMMARY OF THE INVENTION

Accordingly, the present invention provides an improved process for the preparation of cefadroxil of the formula (I)

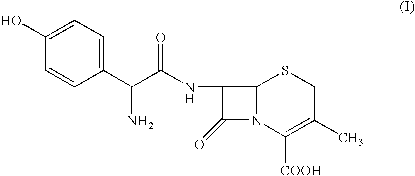

having water content in the range of 4-5 %, which comprises the steps of:

i) silylating the 7-amino desacetoxy cephalosporanic acid (7-ADCA) of the formula (II) with trimethyl silyl chloride and hexamethyl disilazane (HMDS) in the presence of an organic solvent to obtain silylated derivative of 7-ADCA of the formula (III) wherein R₁ represents methyl group at a temperature in the range of 30° C. to reflux temperature of the solvent,

ii) condensing the mixed anhydride of the formula (VI) prepared from Dane salt of formula (IV) wherein R₃ represents methyl, ethyl or isopropyl and M⁺ is sodium or potassium and chloroformate of formula (V) wherein R₂ represents alkyl, phenyl, benzyl or cycloalkyl in the presence of mixture of solvents and a catalyst, with the solution of silylated derivative of 7-ADCA of the formula (III) obtained in step (i) above to produce a compound of formula (VII), wherein R₁ and R₃ are as defined above,

iii) hydrolyzing the compound of formula (VII) using dilute acid,

iv) adding DMF, adjusting the pH of the solution to 4-6 to obtain DMF solvate of cefadroxil,

v) desolvating the cefadroxil DMF solvate in water by heating at a temperature in the range of 30-70° C. for a period of 1 to 4 h, and

vi) cooling the resulting solution to 0 to 20° C. and isolating the product formed to obtain cefadroxil having water content in the range of 4-5%. The process is shown in Scheme-1 below:

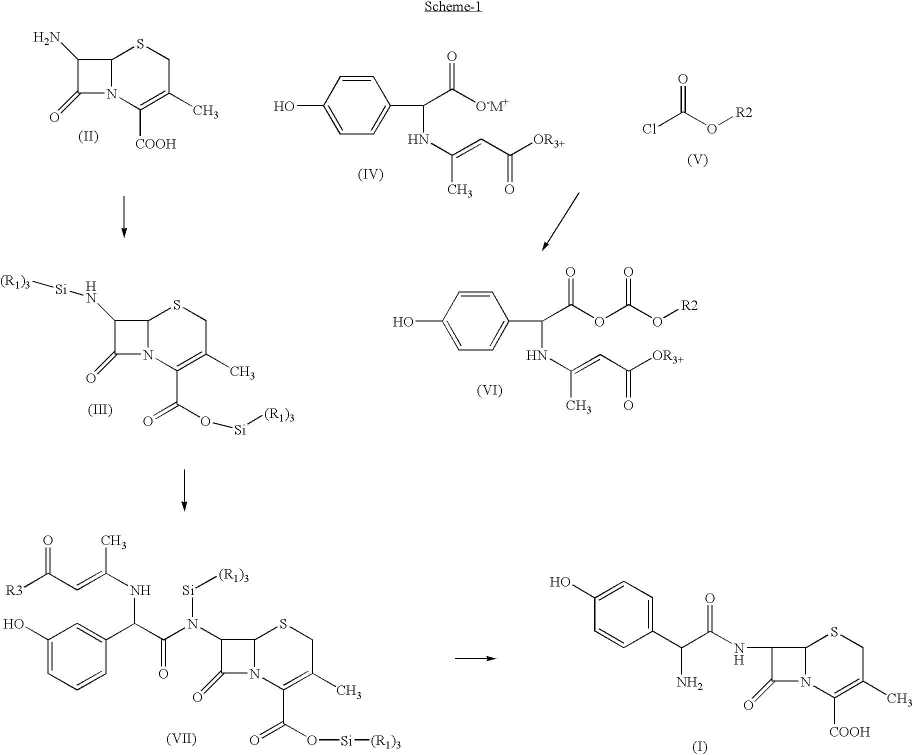

### DETAILED DESCRIPTION OF THE INVENTION

In yet another embodiment of the present invention, the silylation in step (i) is carried out in the presence of solvents such as halogenated hydrocarbons, ethyl acetate, tetrahydrofuran, acetonitrile, N,N-dimethylformamide and the like or mixtures thereof.

In yet another embodiment of the present invention, the solvents used for preparing mixed anhydride may be selected from mixture of MDC/dimethyl acetamide, EDC/dimethyl acetamide, MDC/DMF, EDC/DMF and the like and catalyst such as N-methyl morpholine.

In yet another embodiment of the present invention, the acid used for hydrolysis may be selected from HCl, H₂SO₄ and the like.

In yet another embodiment of the present invention, the pH is adjusted using ammonia.

The advantage of using the combination of HMDS and trimethyl silyl chloride as the silylating agent is that the reaction is faster and the formation of impurities is less.

Another advantage of the process is the use of water for desolvation, wherein the product formed does not contain any other residual solvents except water content in the specific range.

The mixed anhydride of the formula (VI) is prepared from from Dane salt of formula (IV) using a procedure disclosed in the U.S. Pat. No. 4,358,588.

The present invention is provided by the examples given below, which are provided by way of illustration only and should not be considered to limit the scope of the invention.

### EXAMPLE 1

Preparation of 7-[D-α-amino-α-(p-hydroxyphenyl)acetamido]-3-methyl-3-cephem-4-carboxylic acid

To a solution of 7-ADCA (100 gm) in methylene chloride (275 ml), trimethyl silyl chloride (35.6 g) and hexamethyl disilazane (51 g) were added. The reaction mass was stirred for 120-130 minutes at 38-46° C. to get silylated derivative of 7-ADCA, which was condensed with the mixed anhydride of D(−) α-4-hydroxyphenyl glycine Dane salt methyl, potassium, obtained by reaction of D(−) α-4-hydroxyphenyl glycine Dane Salt methyl, potassium (152 g) with methyl chloroformate (48 g) in a mixture of dichloromethane (530 ml) and N,N-dimethylacetamide (170 g), in presence of amount of catalytic N-methyl morpholine (1.4 g) at −44 to −40° C. for 90-100 minutes. After completion of reaction, the reaction mass was subjected to hydrolysis in dilute hydrochloric acid (325 ml, 6.2% aqueous hydrochloric acid). Aqueous layer was separated and to this N,N-dimethylformamide (1050 ml) was added. pH of the solution was adjusted to 5.6-6.0 with dilute ammonia (80 ml) at 24-32° C. to get cefadroxil DMF solvate, which was filtered and washed with aqueous DMF (100 ml) followed by acetone wash (400 ml). Cefadroxil DMF solvate was desolvated in purified water (300 ml) at 38-54° C. for 90-120 minutes. The product slurry was cooled to 10° C. and filtered, washed with acetone (250 ml) and dried to get cefadroxil (157-162 g), water content 4.7%.

1. A process for the preparation of cefadroxil of the formula (I), 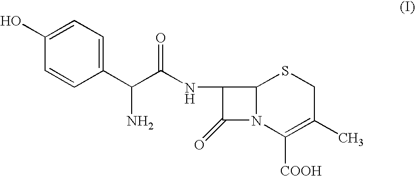
having water content in the range of 4-5%, which comprises the steps of:
i) silylating the 7-amino desacetoxy cephalosporanic acid (7-ADCA) of the formula (II), 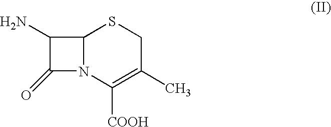
with trimethyl silyl chloride and hexamethyl disilazane (HMDS) in the presence of an organic solvent to obtain silylated derivative of 7-ADCA of the formula (III), 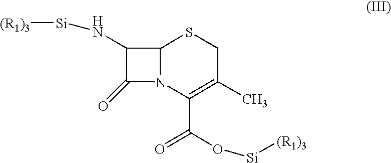
wherein R₁ represents methyl group at a temperature in the range of 30° C. to reflux temperature of the solvent,
ii) condensing the mixed anhydride of the formula (VI) 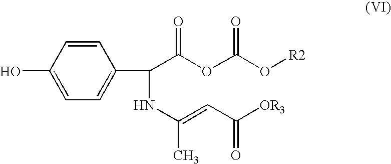
wherein R₂ represents alkyl, phenyl, benzyl or cycloalkyl; R₃ represents methyl, ethyl or isopropyl with the solution of silylated derivative of 7-ADCA of the formula (III) obtained in step (i) above to produce a compound of formula (VII), 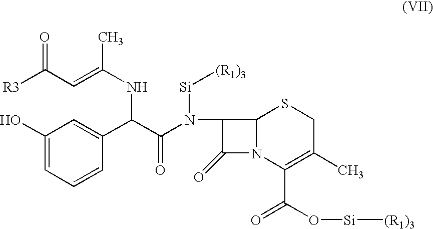
wherein R₁ and R₃ are as defined above,
iii) hydrolyzing the compound of formula (VII) using dilute acid,
iv) adding DMF, adjusting the pH of the solution to 4-6 to obtain DMF solvate of cefadroxil,
v) desolvating the cefadroxil DMF solvate in water by heating at a temperature in the range of 30-70° C. for a period of 1 to 4 h and
vi) cooling the resulting solution to 0 to 20° C. and isolating the product formed to obtain cefadroxil having water content in the range of 4-5%.

2. The process as claimed in claim 1, the solvent used in step (i) is selected from halogenated hydrocarbons, ethyl acetate, tetrahydrofuran, acetonitrile, N,N-dimethylformamide or mixtures thereof.

3. The process as claimed in claim 1, wherein the acid used in step (iii) is selected from HCl or H₂SO₄.

4. The process as claimed in claim 1, wherein in step (iv) the pH is adjusted using ammonia.
